(19) United States
(12) Patent Application Publication
(10) Pub. No.: US 2011/0218440 A1
(43) Pub. Date: Sep. 8, 2011

(54) ULTRASONIC DIAGNOSTIC APPARATUS AND SIGNAL PROCESSING METHOD IN ULTRASONIC DIAGNOSTIC APPARATUS

(75) Inventors: Nobuhiko Fujii, Tokyo (JP); Hajime Otaki, Tokyo (JP)

(73) Assignee: HITACHI MEDICAL CORPORATION, Tokyo (JP)

(21) Appl. No.: 13/129,262

(22) PCT Filed: Nov. 10, 2009
(86) PCT No.: PCT/JP09/69079

§ 371 Date: May 13, 2011

(30) Foreign Application Priority Data
Nov. 14, 2008 (JP) 2008-292372

(51) Int. Cl. A61B 8/14

(52) U.S. Cl. 600/443

(57) ABSTRACT

An ultrasonic diagnostic apparatus of the invention includes: an ultrasonic probe that transmits an ultrasonic beam to a subject and receives reflected echo signals from the subject; a beam forming section that supplies a driving signal causing the ultrasonic probe to transmit the ultrasonic beam; a data conversion section that converts echo data obtained by digitalizing the reflected echo signals into ultrasonic image data; and a display that displays ultrasonic images based on the converted ultrasonic image data, wherein the ultrasonic diagnostic apparatus includes a process configuration text which is capable of rewriting a plurality of signal processes executed by the data conversion section and the execution order of the plurality of signal processes in a general language, and wherein the data conversion section includes a controller that executes the plurality of signal processes on the echo data based on the description of the process configuration text to thereby convert the echo data into the ultrasonic image data.

### TECHNICAL FIELD

The invention relates to an ultrasonic diagnostic apparatus, and more particularly, to a technique for changing a signal process configuration of a data conversion section that converts reflected echo signals from a subject into ultrasonic image data.

### BACKGROUND ART

An ultrasonic diagnostic apparatus transmits an ultrasonic wave to the internal part of a subject using an ultrasonic probe and receives reflected echo signals from the internal part of the subject to generate tomographic images (B-mode images) of the area to be captured of the subject, for example, and displays the B-mode images for diagnostic purposes.

For example, in order to generate and display the B-mode images, the ultrasonic diagnostic apparatus performs various signal processes on the reflected echo signals from the subject. Echo data obtained by digitalizing the reflected echo signals from the subject are subjected to an ultrasonic scanning process, such as a logarithmic compression process and a persistence process, and a TV scanning process, such as a scanning conversion process and a gamma correction process, by a data conversion section, and are displayed.

In addition to B-mode images, the ultrasonic diagnostic apparatus generates various ultrasonic images such as time-varying images (M-mode images) of the capturing area or Doppler images obtained based on a bloodstream velocity or the like of the subject and displays the ultrasonic images for diagnostic purposes. Moreover, the ultrasonic diagnostic apparatus has many capturing modes, for example, such as a B mode wherein only the B-mode images are displayed, a B/M mode wherein B-mode images and M-mode images are generated through parallel processing and displayed on the same screen, and a B/D mode wherein B-mode images and Doppler images are generated through parallel processing and displayed on the same screen.

When generating a plurality of ultrasonic images through parallel processing and displaying the images, for example, as disclosed in PTL 1, a technique in which signal process is performed using a plurality of data processing systems arranged in parallel, such as a memory and a digital scan converter (DSC), is known.

### CITATION LIST

### Patent Literature

[PTL 1] JP-A-8-252253

### SUMMARY OF INVENTION

### Technical Problem

However, as described above, although it is necessary for the data conversion section to execute various signal processes in order to generate and display ultrasonic images, the flexibility to change a signal process configuration executed by the data conversion section is not considered in the technique of PTL 1.

That is, in the data conversion section, a signal process configuration (signal processes executed, an execution order of signal processes executed) unique to a capturing mode is set in advance, and various signal processes are executed in accordance with the signal processing configuration. However, it is preferable to flexibly change the signal processing configuration.

For example, in a development stage of the ultrasonic diagnostic apparatus, in some cases, the performance of the system is verified by checking the change in the appearance of ultrasonic images while changing the execution order of the respective signal processes and adding or deleting a signal process without changing the content itself of the respective signal processes. Moreover, in a maintenance stage of the ultrasonic diagnostic apparatus, in some cases, the signal processing configuration is changed in accordance with a client demand or the like. However, whenever the signal processing configuration is changed in such a case, it takes a long time to rewrite, recompile, and execute the source codes of the program language by which the content of the respective signal processes, the execution order, and the like are described. Thus, it is not desirable from the perspective of development efficiency and maintenance efficiency.

Therefore, an object of the invention is to improve the flexibility to change a signal process configuration of the data conversion section.

### Solution to Problem

An ultrasonic diagnostic apparatus of the invention is configured to include: an ultrasonic probe that transmits an ultrasonic beam to a subject and receives reflected echo signals from the subject; a beam forming section that supplies a driving signal causing the ultrasonic probe to transmit the ultrasonic beam; a data conversion section that converts echo data obtained by digitalizing the reflected echo signals into ultrasonic image data; and a display that displays ultrasonic images based on the converted ultrasonic image data.

Particularly, in order to solve the above-described problems, the ultrasonic diagnostic apparatus includes a process configuration text which is capable of rewriting a plurality of signal processes executed by the data conversion section and the execution order of the plurality of signal processes in a general language, and the data conversion section executes the plurality of signal processes on the echo data based on the description of the process configuration text to thereby convert the echo data into the ultrasonic image data.

A signal processing method in the ultrasonic diagnostic apparatus of the invention includes: a step wherein an ultrasonic probe transmits an ultrasonic beam to a subject and receives reflected echo signals from the subject; a step wherein a beam forming section supplies a driving signal causing the ultrasonic probe to transmit the ultrasonic beam; a step wherein a data conversion section converts echo data obtained by digitalizing the reflected echo signals into ultrasonic image data; a step wherein a display displays ultrasonic images based on the converted ultrasonic image data; and a step wherein a controller has a process configuration text which is capable of rewriting a plurality of signal processes executed by the data conversion section and the execution order of the plurality of signal processes in a general language, and the data conversion section executes the plurality of signal processes on the echo data based on the description of the process configuration text to thereby convert the echo data into the ultrasonic image data.

According to this configuration, when it is necessary to change the signal processing configuration, it is possible to change the signal processing configuration by only rewriting the process configuration text described in a general language without rewriting the source code which describes the content itself of the respective signal processes in a program language.

More specifically, the process configuration text may be configured to describe a plurality of signal process names executed by the data conversion section and an input buffer name and an output buffer name of each of the plurality of signal process names, and an output buffer name of one signal process name may be made identical to an input buffer name of another signal process name to thereby describe the execution order of a signal process associated to the one signal process name and a signal process associated to the another signal process name.

According to this configuration, when it is desired to change the execution order of the respective signal processes, for example, it is sufficient to rewrite the input buffer name and the output buffer name to thereby change the interconnection information of the execution order between the signal processes. Moreover, when it is desired to delete part of the signal processes, it is sufficient to delete a signal process name corresponding to the signal process to be deleted to thereby rewrite the input and output buffer names so as to comply with the deletion. Furthermore, when it is desired to add a signal process, it is sufficient to add a signal process name corresponding to the signal process to be added to thereby rewrite the input and output buffer names so as to comply with the addition.

In this way, by using the names of signal processes used in the ultrasonic diagnostic apparatus and the process configuration text which can be rewritten in a general language and which includes an input buffer name and an output buffer name corresponding to each of the signal process names, and the like, it is possible to flexibly change the signal processing configuration. The execution content itself of the respective signal processes is set in advance, and the process configuration text describes signal processes which will be executed among a plurality of signal processes set in advance and the execution order thereof.

However, when the data conversion section is configured to include a plurality of processors, the process configuration text may be configured to describe a plurality of signal process block names, a plurality of signal process names executed by the data conversion section, of each of the plurality of signal process block names, and an input buffer name and an output buffer name of each of the plurality of signal process names, and a signal process group associated to the plurality of signal process names in the respective signal process block names may be allocated to the plurality of processors and executed in parallel.

As described above, in order to generate and display ultrasonic images, an ultrasonic scanning process such as a logarithmic compression process and a persistence process and a TV scanning process such as a scanning conversion process and a gamma correction process are required. Since in some cases, since the ultrasonic scanning process and the TV scanning process have different processing rates, if these processes are executed by the same processor, the process becomes lossy, and it is not desirable from the perspective of processing efficiency. Therefore, by allocating signal processes having different processing rates to a plurality of processors of the data conversion section, for example, so as to be subjected to parallel processing, it is possible to improve the processing efficiency. Here, as in the invention, by configuring the process configuration text to describe signal process block names to thereby describe a signal process group to be executed in parallel for each signal process block name, it is possible to easily describe parallel signal processes.

Moreover, the process configuration text may be configured to be capable of describing a synchronization name for a pair of signal process names which is executed in synchronization between the signal process group associated to one signal process block name and the signal process group associated to another signal process block name, and signal processes associated to a signal process name for which the synchronization name is described may be executed in synchronization with each other.

That is, the ultrasonic diagnostic apparatus has various capturing modes, and when generating B-mode images and Doppler images in parallel in a B/D mode, for example, and combining and displaying these images, it is necessary to synchronize the two images in the combining process. Here, as in the invention, by enabling the synchronization names to be described for a pair of signal process names executed in synchronization, it is possible to easily describe synchronization between parallel processes.

Moreover, the process configuration text may be configured to be capable of describing a table name for the signal process name, in which process coefficients used in signal processes are stored, and a process coefficient associated to the table name may be referenced when executing a signal process associated to a signal process name for which the table name is described.

### Advantageous Effects of Invention

According to the invention, it is possible to improve the flexibility to change a signal process configuration of the data conversion section.

### BRIEF DESCRIPTION OF DRAWINGS

FIG. 1 is a block diagram showing a configuration of an ultrasonic diagnostic apparatus to which the invention is applied.

FIG. 2 is a diagram showing a process flow up to a state when B-mode images are generated using one processor and output to a video memory.

FIG. 3 is a diagram showing a description example of a process configuration text of a first embodiment.

FIG. 4 is a diagram showing a process flow up to a state when B-mode images are generated using two processors and output to a video memory.

FIG. 5 is a diagram showing a description example of a process configuration text of a second embodiment.

FIG. 6 is a diagram showing a process flow up to a state when B-mode images and Doppler images are generated using four processors and output to a video memory.

FIG. 7 is a diagram showing a description example of a process configuration text of a third embodiment.

### DESCRIPTION OF EMBODIMENTS

Hereinafter, embodiments of an ultrasonic diagnostic apparatus to which the invention is applied will be described. In the following description, components having the same functions will be denoted by the same reference numerals, and redundant description thereof will be omitted.

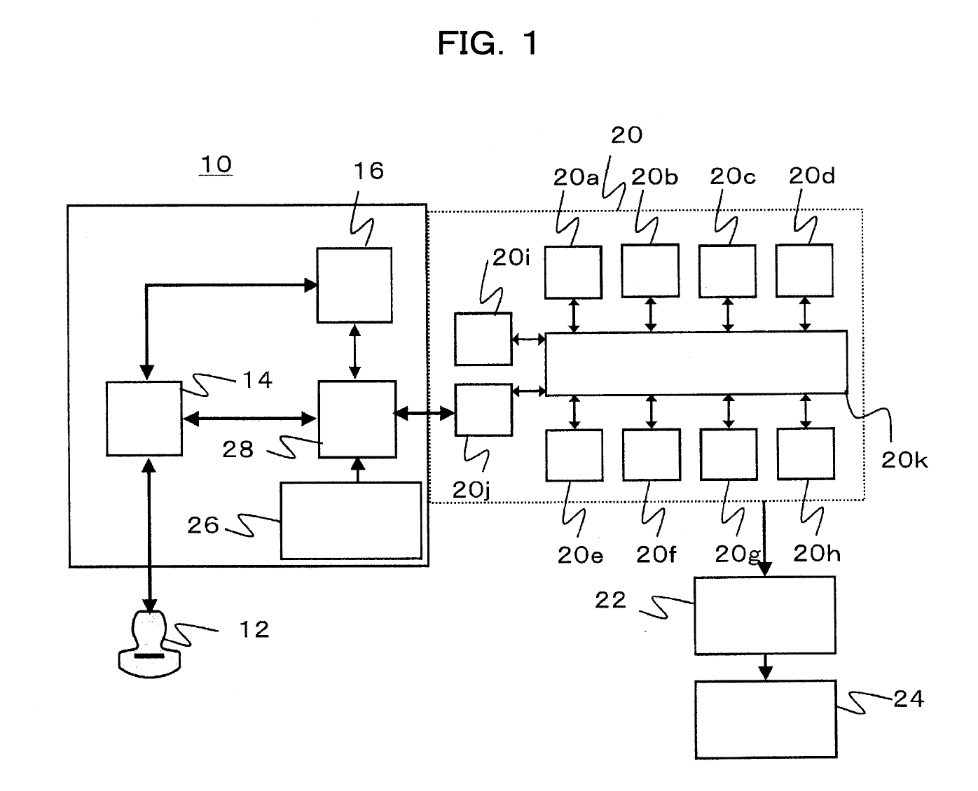

FIG. 1 is a block diagram showing a configuration of an ultrasonic diagnostic apparatus to which the invention is applied. As shown in FIG. 1, an ultrasonic diagnostic apparatus 10 of the invention is configured to include an ultrasonic probe 12 (PROBE) that transmits an ultrasonic beam to a subject and receives reflected echo signals from the subject, a transmission/reception switching section 14 (PRB) that switches transmission/reception of the ultrasonic probe 12, a beam forming section 16 (DBF) that supplies a signal for transmitting the ultrasonic beam to the ultrasonic probe 12, a data conversion section 20 that performs a plurality of signal processes on echo data obtained by digitalizing the reflected echo signals from the subject to convert the echo data into ultrasonic image data, a video memory 22 that stores ultrasonic images and the like based on the ultrasonic image data converted by the data conversion section 20, a display 24 that displays the ultrasonic images and the like stored in the video memory 22, and other components.

The ultrasonic diagnostic apparatus 10 also includes a setting section 26 (CONSOLE) that sets capturing mode data of the ultrasonic image data to the echo data, and a controller 28 (CONT) that collates the capturing mode data and the echo data to generate a data set, and transmits the data set to the data conversion section 20 or controls the data conversion section 20 to analyze the capturing mode data in the data set to thereby convert the echo data in the data set into ultrasonic image data using the analyzed capturing mode data.

The ultrasonic probe 12 (PROBE) has vibrators which are arranged in the long-axis direction of the ultrasonic probe so as to correspond to the first to m-th channels. Here, when the vibrators are divided into k channels in the short-axis direction and arranged so as to correspond to the first to k-th channels, by changing the delay time applied to the respective vibrators (first to k-th channels) in the short-axis direction, the transmission and reception beams can also be focused in the short-axis direction. Moreover, when the amplitude of ultrasonic transmission signals applied to the respective vibrators in the short-axis direction is changed, the transmission weighting is realized. On the other hand, when the amplification or attenuation level of ultrasonic reception signals from the respective vibrators in the short-axis direction is changed, the reception weighting is realized. Furthermore, when each of the vibrators in the short-axis direction is turned ON and OFF, an aperture can be controlled.

The ultrasonic probe 12 may have vibrators which are formed of a piezoelectric element. Moreover, the ultrasonic probe 12 may have vibrators which are formed of a semiconductor called a cMUT (Capacitive Micromachined Ultrasonic Transducer: see, for example, IEEE Trans. Ultrason. Ferroelect. Freq. Contr. Vol. 45, pp. 678-690, May 1998).

The transmission/reception switching section 14 performs the function of an interface that supplies a transmission signal to the ultrasonic probe 12 and processes the received reflected echo signals. Moreover, the transmission/reception switching section 14 has the function of a reception circuit that receives the reflected echo signals from the internal part of the subject in response to an ultrasonic beam transmitted to the subject and collects biological information.

The beam forming section 16 is a transmission circuit that controls the ultrasonic probe 12 to output an ultrasonic beam, and is configured to control the transmission time of ultrasonic pulses for driving a plurality of vibrators of the ultrasonic probe 12 to form an ultrasonic beam toward a focal point set within the subject. Moreover, the beam forming section 16 is configured to electronically scan the ultrasonic beam in the arrangement direction of the vibrators of the ultrasonic probe.

The data conversion section 20 is configured to perform various signal processes described later on the echo data which have been received by the transmission/reception switching section 14 and subjected to a reception process such as amplification, A/D conversion, a process of aligning the phases between a plurality of vibrators and adding the phase signals, and the like to thereby convert the echo data into ultrasonic tomographic image data, and forms ultrasonic images based on the echo data which are input sequentially. Examples of the ultrasonic images include an A-mode image, a B-mode image, a color flow mapping (C)-mode image, a Doppler (D)-mode image, an elastic (E)-mode image, an M-mode image, a 3D ultrasonic image which is generally obtained by reconstructing the B-mode images arranged continuously along the body surface of the subject.

The video memory 22 stores the ultrasonic images formed by the data conversion section 20 by combining the same with the character or graphic information such as patient information or body mark information, and the graphic information set by the setting section 26. The controller 28 also has the function of a display controller that selects and controls the display format to be used for displaying images.

The display 24 displays the ultrasonic images stored in the video memory 22, and is formed, for example, of a CRT monitor or a liquid crystal monitor. The display 24 may only need to be capable of displaying ultrasonic images and display images from which an operator can make a diagnosis, and the invention can be applied to any display whether it outputs analog signals or digital signals.

The setting section 26 allows an operator to input various parameters such as a desired capturing mode, patient information, and a capturing position using a keyboard and a trackball on a console.

The controller 28 is a control computer system for controlling the transmission/reception switching section 14, the beam forming section 16, and the data conversion section 20 to function appropriately based various parameters input by the setting section 26.

As shown in FIG. 1, the data conversion section 20 includes a plurality of processors 20a to 20h, a processor controller 20i that collectively controls the processors 20a to 20h, an internal memory 20j (MEMORY) that records echo data transmitted through the transmission/reception switching section 14, and an internal data transfer bus 20k that is capable of communicating data with the processors 20a to 20h, the processor controller 20i, and the internal memory 20j.

The internal memory 20j receives and stores data transmitted from the controller 28 (CONT). The processor controller 20i controls the processors 20a to 20h connected through the internal data transfer bus 20k. As a specific example of the control, the processor controller 20i analyzes the data and parameters stored in the internal memory 20j and allocates a processing program to the processors 20a to 20h to thereby obtain ultrasonic images from the data stored in the internal memory 20j.

The data conversion section 20 may use a multicore processor, for example, CELL, which is a processor standardized by an architecture in which a plurality of processor cores is integrated in a single package. The CELL is an abbreviation of Cell Broadband Engine (registered trademark) and is a microprocessor developed by Sony Computer Entertainment Corporation and the like. When using CELL, the processors 20a to 20h are configured as SPE (Synergistic Processor Element), the processor controller 20i is configured as PPE (PowerPC Processor Element), and the internal data transfer bus 20k is configured as EIB (Element Interconnect Bus).

In ultrasonic diagnostic apparatuss, there is a demand to decrease the circuit size of a data processor contributing to miniaturization of the system and a demand to increase the data processing speed of the data processor contributing to performance of the system. There is still a technical problem that the two demands are in a trade-off relationship. However, the use of a single-package multicore processor can contribute to decreasing the circuit size and increasing the data processing speed. Moreover, although the number of processors is eight as an example in FIG. 1, since the number can be arbitrarily set in accordance with the processing capability of the processor, the number may be an arbitrary natural number.

However, although the detail will be described later, in the data conversion section 20 of the ultrasonic diagnostic apparatus 10, a signal process configuration (signal processes executed, an execution order of signal processes executed) unique to a capturing mode is set in advance, and processes are executed in accordance with the signal processing configuration depending on a capturing mode. Here, for example, in a development stage of the system, in some cases, the performance of the system is verified by checking the change in the appearance of ultrasonic images while changing the execution order of the respective signal processes and adding or deleting a signal process without changing the content itself of various signal processes in the data conversion section 20. Moreover, in a maintenance stage of the ultrasonic diagnostic apparatus, in some cases, the signal processing configuration is changed in accordance with a client demand or the like.

However, whenever the signal processing configuration is changed in such a case, it takes a long time to rewrite, recompile, and execute the source codes of the program language by which the content of the respective signal processes, the execution order, and the like are described. Thus, it is not desirable from the perspective of development efficiency and maintenance efficiency.

Therefore, the ultrasonic diagnostic apparatus 10 of the present embodiment includes a process configuration text which is capable of rewriting a plurality of signal processes executed by the data conversion section 20 and the execution order of the plurality of signal processes into a general language. The data conversion section 20 is configured to execute the plurality of signal processes on the echo data based on the description of the process configuration text 30 to thereby convert the echo data into the ultrasonic image data. Hereinafter, embodiments of the description of the process configuration text 30 for each capturing mode will be described.

### First Embodiment

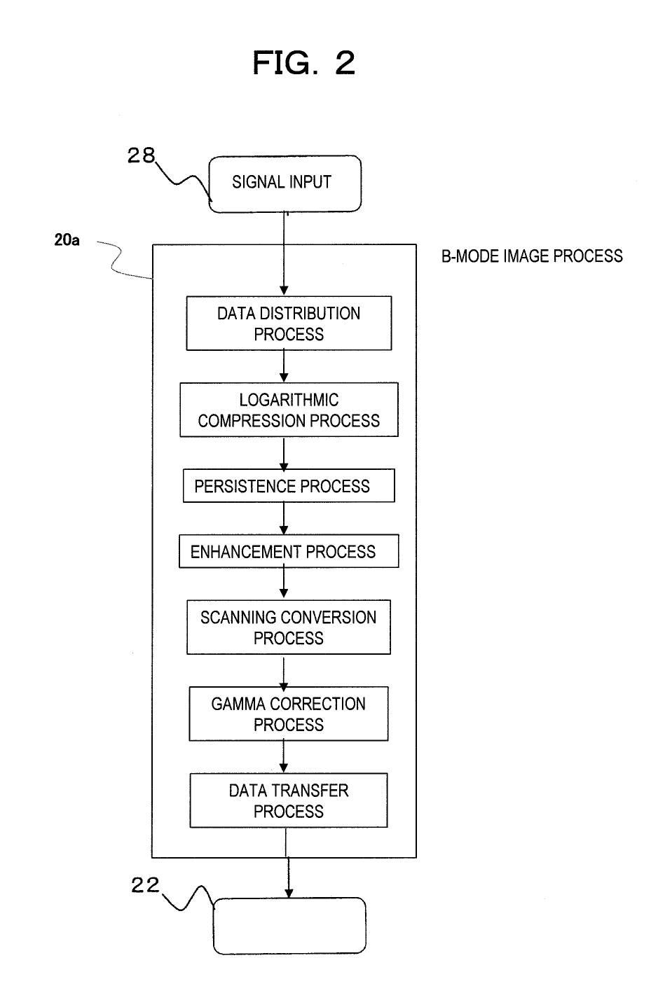

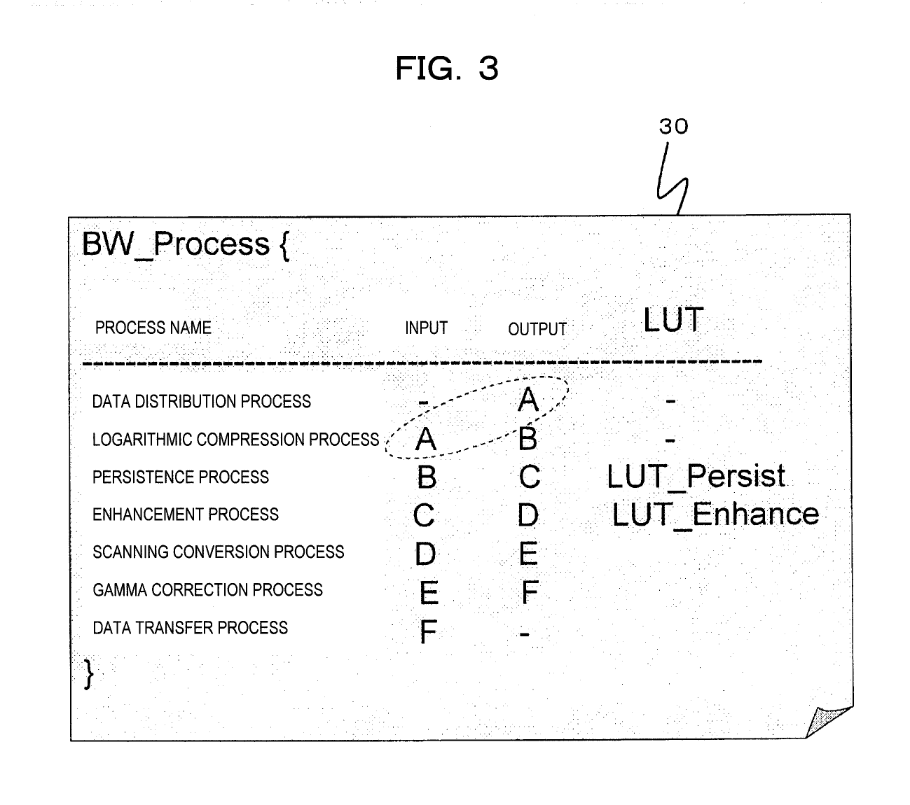

A first embodiment is a description example of the process configuration text when signal processes are executed by one processor of the data conversion section 20. FIG. 2 is a diagram showing a process flow up to a state when B-mode images are generated using one processor and output to the video memory 22. FIG. 3 is a diagram showing a description example in which signal processes of the present embodiment are described in the process configuration text.

As shown in FIG. 2, processes necessary up to the generation and presentation of B-mode images roughly include a data distribution process, a logarithmic compression process, a persistence process, an enhancement process, a scanning conversion process, a gamma correction process, and a data transfer process. These signal processes are executed by being allocated to any one of the processors 20a to 20h by the processor controller 20i.

In the present embodiment, it is assumed that the signal processes are allocated to the processor 20a.

The data distribution process involves determining whether processing target echo data input through the controller 28 are B-mode data and loading the processing target echo data into the processor 20a based on the determination. The determination of the echo data can be performed by the data conversion section 20 analyzing data representing a beam type that is set so as to be associated with the echo data.

The logarithmic compression process involves compressing the dynamic range of ultrasonic reception signals, which is 2 to the 20th power, for example, to a relatively small dynamic range on the circuit, substantially the dynamic range of a monitor. The persistence process involves averaging pixel data which have been subjected to the logarithmic compression process and are displayed at the same position on a monitor. The enhancement process involves enhancing the edges of the pixels having been subjected to the persistence process so that the boundaries between the pixels become definite.

The scanning conversion process involves converting the coordinates of the pixels having been subjected to the enhancement process from the scanning coordinates of the ultrasonic beam to the scanning coordinates on a display monitor. The gamma correction process involves correcting the display gradation of the pixels having been subjected to the scanning conversion process based on a gamma curve that determines the domain of definition of the pixels and the codomain. The data transfer process involves transferring images (B-mode images) obtained through the gamma correction process to the video memory 22.

Next, a method of causing the data conversion section 20 to execute these processes will be described with reference to FIG. 3.

As shown in FIG. 3, BW_Process is described in the process configuration text 30 as a signal process block name, and a description within this signal process block is bound between the parentheses { }. In the signal process block, the names of the above-described respective signal processes are described in the processing order in the signal process name column.

Moreover, in the input and output columns, input and output buffer names are described for each of the signal process names as an interconnection for connecting the respective signal processes. For example, if the output buffer of the logarithmic compression process is B, and the input of the persistence process is the buffer B, the buffer B processed by the logarithmic compression process becomes the input of the persistence process, and the processes are connected as a pipeline process. In other words, the output buffer name (B) of one signal process name (logarithmic compression process) is made identical to the input buffer name (B) of another signal process name (persistence process) to thereby describe the execution order of a signal process associated with one signal process name and a signal process associated with another signal process name.

According to this description, when BW_Process is executed, the respective signal processes are sequentially executed on the echo data based on the description of the process configuration text 30. The content of the respective signal processes is set in advance, and the signal processes described in the process configuration text 30 are executed in the order based on the description.

Moreover, in the LUT column, a table name (LUT: Look Up Table) in which process coefficients used in the signal processes are stored is described for the described signal process name. In the present embodiment, since the persistence process and the enhancement process need to reference LUTs, a table name LUT_Persist is described for the persistence process, and a table name LUT_Enhance is described for the enhancement process. In this way, it is possible to use LUTs prepared in advance, and the table LUT_Persist is referenced when executing the persistence process for which a table name is described, and the table LUT_Enhance is referenced when executing the enhancement process.

As described above, in the process configuration text 30, a plurality of signal processes executed by the data conversion section 20 and the execution order of the plurality of signal processes are described so as to be rewritable in a general language. The general language includes a signal process block name for grasping a plurality of signal processes as one block, a signal process name used for an ultrasonic diagnostic apparatus, an input and output buffer names serving as interconnection information for representing the execution order of the respective signal processes, and a table name in which process coefficients used for the signal process are stored, and the like as described in the present embodiment.

According to the present embodiment, the signal process block name, the respective description input columns, the parentheses { }, and the like are described as a format in advance. By describing only the signal process name and the interconnection information using the input and output buffer names representing the execution order of the signal processes, it is possible to cause a certain signal process group to be processed by a certain processor of the data conversion section 20. Therefore, if the execution order of the signal processes is changed, it is sufficient to rewrite the interconnection using the input and output buffer names in the process configuration text 30.

Moreover, when it is desired to add a signal process, it is sufficient to add a signal process name corresponding to the signal process name to be added to the process configuration text 30 and rewrite the interconnection information using the input and output buffer names so as to comply with the addition. Furthermore, when it is desired to delete a signal process, it is sufficient to delete a signal process name corresponding to the signal process name to be deleted from the process configuration text 30 and rewrite the interconnection information using the input and output buffer names so as to comply with the deletion. As a result, by rewriting the description of the process configuration text without changing the source code itself of the data conversion section 20, described in a program language, for example, it is possible to easily change the signal processing configuration.

In this way, for example, in a development stage of an ultrasonic diagnostic apparatus, when it is desired to verify the performance of the system while appropriately changing the signal processing configuration, it is possible to flexibly change the signal processing configuration.

For example, it is possible to easily verify how the appearance of ultrasonic images will be changed when the order of signal processes is changed. Moreover, since the signal processing configuration can be viewed as a text, the overall structure of the signal processing configuration can be easily grasped. As a result, the development efficiency of the ultrasonic diagnostic apparatus can be improved. Moreover, for example, in a maintenance stage of the ultrasonic diagnostic apparatus, since the signal processing configuration can be changed flexibly in accordance with a client demand or the like, the maintenance efficiency can be improved.

### Second Embodiment

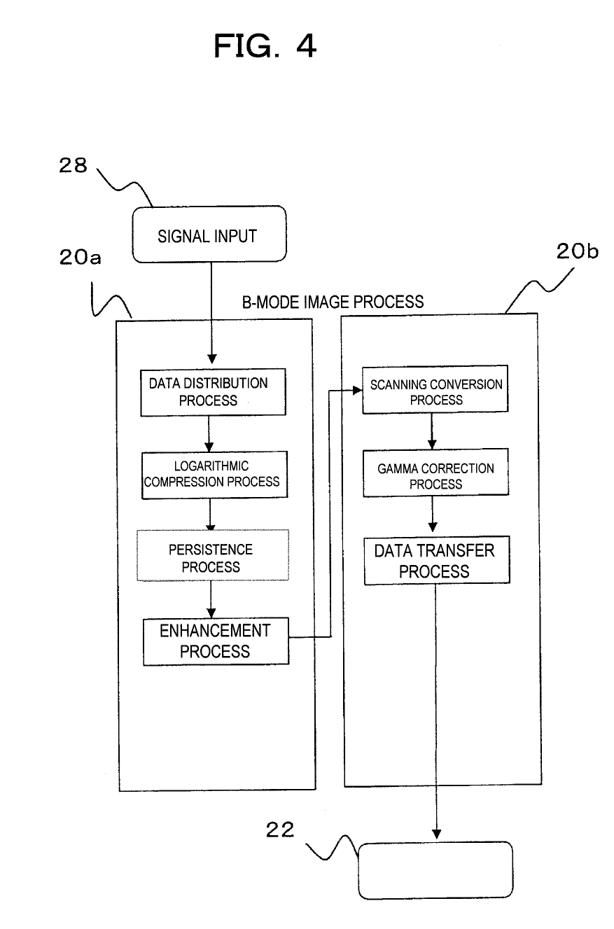

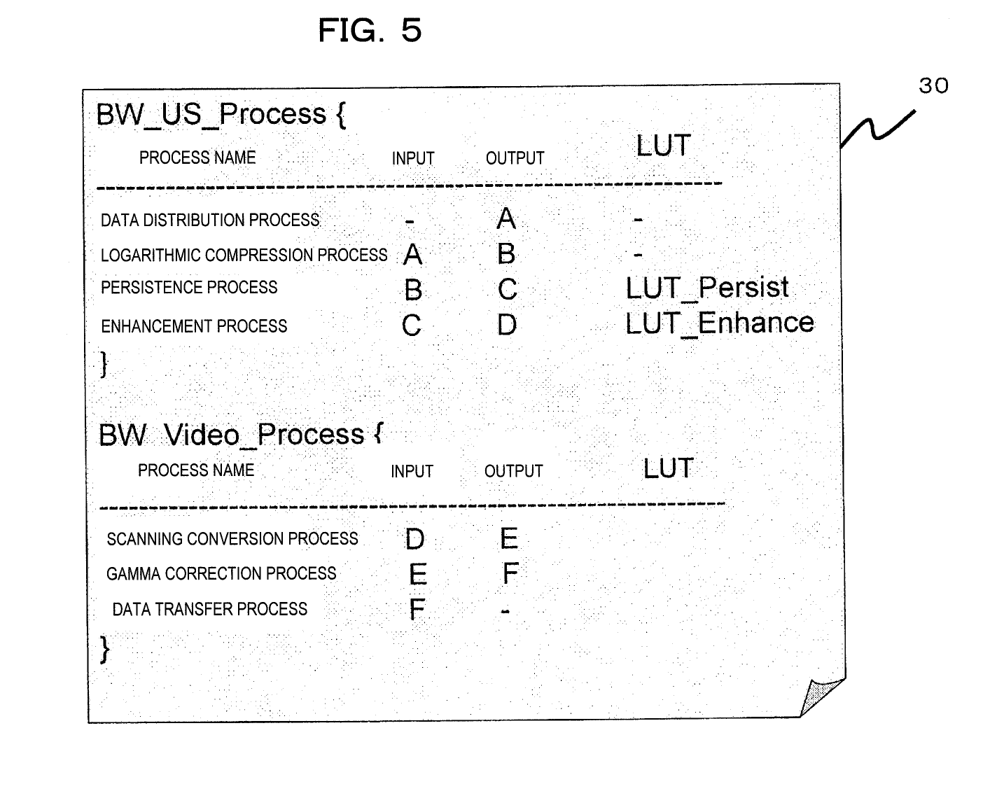

A second embodiment is a description example of the process configuration text when signal processes are executed in parallel by two processors of the data conversion section 20. FIG. 4 is a diagram showing a process flow up to a state when B-mode images are generated using two processors and output to the video memory 22. FIG. 5 is a diagram showing a description example in which signal processes of the present embodiment are described in the process configuration text.

As shown in FIG. 4, similarly to the first embodiment, processes necessary up to the generation and presentation of B-mode images roughly include a data distribution process, a logarithmic compression process, a persistence process, an enhancement process, a scanning conversion process, a gamma correction process, and a data transfer process. The difference from the first embodiment is that these signal processes are executed by being allocated to any two of the processors 20a to 20h by the processor controller 20i.

In this respect, as shown in FIG. 4, the B-MODE IMAGE PROCESS can be divided into au ultrasonic scanning process which includes a data distribution process, a logarithmic compression process, a persistence process, and an enhancement process, and a TV scanning process which includes a scanning conversion process, a gamma correction process, and a data transfer process. The ultrasonic scanning process is allocated to any one of the processors 20a to 20h by the processor controller 20i, and the TV scanning process is allocated to any one of the processors 20a to 20h which are not allocated to the ultrasonic scanning process. In the present embodiment, it is assumed that the ultrasonic scanning process is allocated to the processor 20a (BM1) and the TV scanning process is allocated to the processor 20b (BM2).

Since the ultrasonic scanning process and the TV scanning process have different processing rates, it is preferable to execute the processes by allocating them to different processors from the perspective of processing efficiency. Moreover, since these two scanning processes can be processed exclusively, the processing speed can be increased by executing the processes in parallel by a pipeline method.

Next, a method of causing the data conversion section 20 to execute these processes will be described with reference to FIG. 5.

As shown in FIG. 5, BW_US_Process and BW_Video_Process are described in the process configuration text 30 as signal process block names, and descriptions within these signal process blocks are bounded between the parenthesis { }. In each of the signal process blocks, the names of the above-described respective signal processes are described in the processing order in the signal process name column. That is, a data distribution process, a logarithmic compression process, a persistence process, and an enhancement process are described in the BW_US_Process block, and a scanning conversion process, a gamma correction process, and a data transfer process are described in the BW_Video_Process block.

Moreover, in the input and output columns, input and output buffer names are described for each of the signal process names as an interconnection for connecting the respective signal processes. For example, signal processes can be also connected between the signal process blocks so that the output buffer of the enhancement process is D and the input of the scanning conversion process is the buffer D. In this way, the buffer D processed by the enhancement process becomes the input of the scanning conversion process, and the processes are connected as a pipeline process.

According to the present embodiment, by describing the signal process block name, the respective description input columns, the parenthesis { }, and the like as a format in advance, and describing only the signal process name and the interconnection information using the input and output buffer names representing the execution order of the signal processes, it is possible to cause a plurality of signal process groups to be processed by a plurality of processors of the data conversion section 20.

That is, signal process groups (an ultrasonic scanning process group and a TV scanning process group) associated to a plurality of signal process names in the respective signal process block names are allocated to a plurality of processors (the processors 20a and 20b) and executed in parallel. When it is desired to cause a plurality of signal process groups to be executed in parallel as described above, by dividing the signal process block name in the process configuration text 30 and describing a plurality of signal process blocks, it is possible to easily change the signal processing configuration without changing the source code itself of the data conversion section 20, described in a program language, for example.

In this way, for example, in a development stage of an ultrasonic diagnostic apparatus, when it is desired to verify the performance of the system while appropriately changing the signal processing configuration, it is possible to flexibly change the signal processing configuration. Moreover, since the signal processing configuration can be viewed as a text, the overall structure of the signal processing configuration can be easily grasped. As a result, the development efficiency of the ultrasonic diagnostic apparatus can be improved. Moreover, for example, in a maintenance stage of the ultrasonic diagnostic apparatus, since the signal processing configuration can be changed flexibly in accordance with a client demand or the like, the maintenance efficiency can be improved.

Particularly, as in the present embodiment where the data conversion section 20 has a plurality of processors, in order to effectively drive the plurality of processors in parallel, it is necessary to perform performance evaluation of the system while changing the signal processing configuration in various ways while considering the processing rates or the like of the respective signal processes. In this respect, as in the present embodiment, when causing a plurality of processors to perform signal processes in parallel, it is possible to easily describe a parallel signal process in the process configuration text 30 and to easily change the signal processing configuration. Thus, it is possible to improve the development efficiency of the ultrasonic diagnostic apparatus.

### Third Embodiment

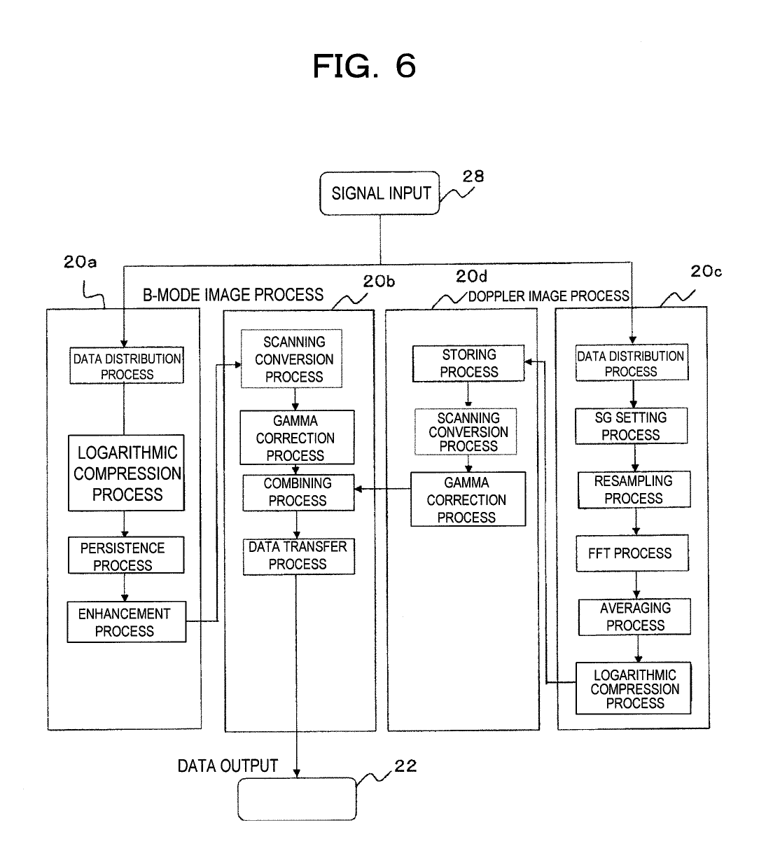

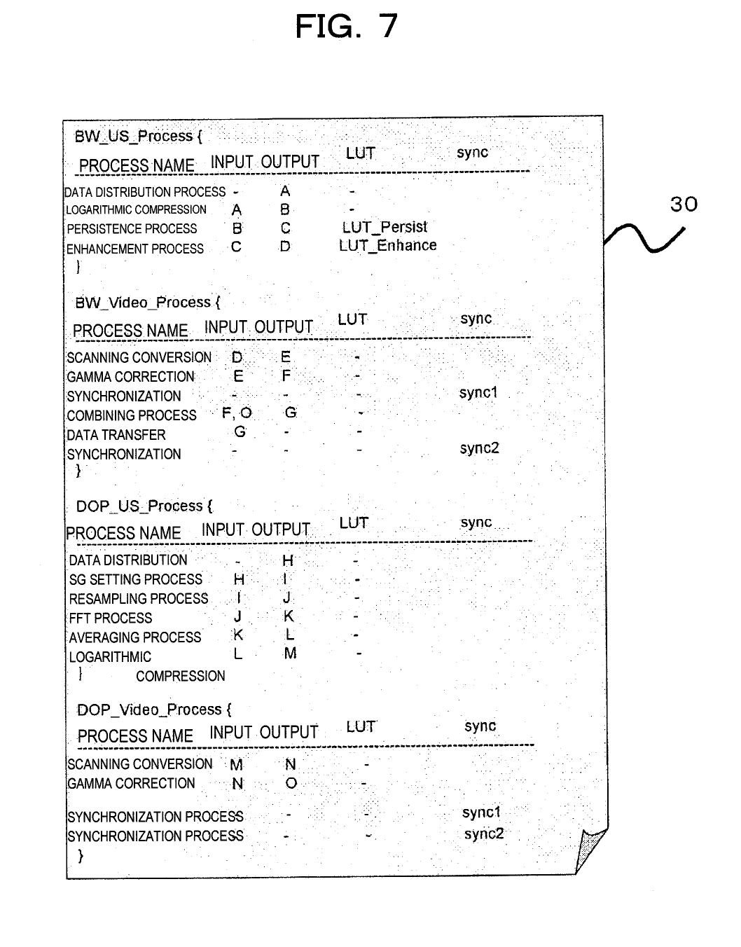

A third embodiment is a description example of the process configuration text when signal processes are executed in parallel by four processors of the data conversion section 20, and the parallel processes are synchronized. FIG. 6 is a diagram showing a process flow up to a state when B-mode images and Doppler images are generated using four processors and output to the video memory 22. FIG. 7 is a diagram showing a description example in which signal processes of the present embodiment are described in the process configuration text.

As shown in FIG. 6, in the present embodiment, B-mode images and Doppler images are generated and presented as combined images. Description of the same processes necessary up to the generation and presentation of the B-mode images as those of the second embodiment will be omitted, and only the difference will be described. The TV scanning process of B-mode images includes a combining process which is inserted between the gamma correction process and the data transfer process. The combining process involves combining data which have been subjected to a TV scanning process of Doppler images described later with B-mode images. Moreover, the data transfer process involves transferring images (combined images of B-mode images and Doppler images) obtained through the combining process to the video memory 22.

The processes necessary up to the generation and presentation of Doppler images roughly include a data distribution process, a sample gate (SG) setting process, a resampling process, a fast Fourier transform (FFT) process, an averaging process, a logarithmic compression process, a storing process, a scanning conversion process, and a gamma correction process.

These respective processes can be divided into an ultrasonic scanning process which includes the data distribution process, the SG setting process, the resampling process, the fast Fourier transform (FFT) process, the averaging process, and the logarithmic compression process, and a TV scanning process which includes the storing process, the scanning conversion process, and the gamma correction process.

For each of the B-mode images and the Doppler images, the ultrasonic scanning process is allocated to any one of the processors 20a to 20h by the processor controller 20i, and the TV scanning process is allocated to any one of the processors 20a to 20h which are not allocated to the ultrasonic scanning process. In the present embodiment, it is assumed that the ultrasonic scanning process of B-mode images is allocated to the processor 20a (BM1) and the TV scanning process is allocated to the processor 20b (BM2).

Moreover, it is assumed that the ultrasonic scanning process of Doppler images is allocated to the processor 20c (DM1) and the TV scanning process is allocated to the processor 20d (DM2).

That is, as for Doppler images, since the ultrasonic scanning process and the TV scanning process have different processing rates, it is preferable to execute the processes by allocating them to different processors from the perspective of processing efficiency. Moreover, since these two scanning processes can be processed exclusively, the processing speed can be increased by executing the processes in parallel by a pipeline method.

The data distribution process involves determining whether processing target echo data input through the controller 28 are D-mode data and loading the processing target echo data into the processor 20c based on the determination. The determination of the echo data can be performed by the data conversion section 20 analyzing data representing a beam type that is set so as to be associated with the echo data.

The SG setting process involves setting SG to a desired blood velocity diagnostic area on the ultrasonic B-mode images of a subject. The resampling process involves interpolating and calculating sample points which will be computed in Fourier transform at the later stage. The FFT process involves performing frequency analysis on the sample points interpolated by the resampling process and deleting clutter components having relative low frequencies from reflecting bodies having low moving speed such as the myocardium to thereby extract bloodstream component having relatively high frequencies. The averaging process involves obtaining so-called correlation values for each sample point of the bloodstream components extracted by the FFT process. The logarithmic compression process involves compressing the dynamic range of the results of the averaging process to substantially the dynamic range of a monitor.

The storing process involves storing the results of the logarithmic compression process in the internal memory 20j. The scanning conversion process involves converting the coordinate of the pixels stored in the internal memory 20j from the scanning coordinates of the ultrasonic beam to the scanning coordinates on a display monitor. The gamma correction process involves correcting the display gradation of the pixels having been subjected to the scanning conversion process based on a gamma curve that determines the domain of definition of the pixels and the codomain and outputting the corrected pixels to the combining process of B-mode images.

Here, since the process BM1 which is the ultrasonic scanning process of the B-MODE IMAGE PROCESS and the process DM1 which is the ultrasonic scanning process of the Doppler image process need to be operated as different process units, they do not need to be synchronized. However, since the process BM2 which is the TV scanning process of the B-MODE IMAGE PROCESS and the process DM2 which is the TV scanning process of the Doppler image process need to be output to the video memory 22 as the same process unit, they need to be synchronized.

Next, a method of describing the process configuration text 30 in order to realize synchronization between the processors of the data conversion section such as the processors for the B-mode TV scanning process and the D-mode TV scanning process, namely synchronization between process units will be described with reference to FIG. 7. As shown in FIG. 7, BW_US_Process, BW_Video_Process, DOP_US_Process, and DOP_Video_Process are described in the process configuration text 30 as signal process block names, and the descriptions within these signal process blocks are bounded between the parenthesis { }. In each of the signal process blocks, the names of the above-described respective signal processes are described in the processing order in the signal process name column.

That is, a data distribution process, a logarithmic compression process, a persistence process, and an enhancement process are described in the BW_US_Process block, and a scanning conversion process, a gamma correction process, and a data transfer process are described in the BW_Video_Process block. Moreover, a data distribution process, a SG setting process, a resampling process, a FFT process, an averaging process, and a logarithmic compression process are described in the DOP_US_Process block, and a scanning conversion process and a gamma correction process are described in the DOP_Video_Process block.

Moreover, in the input and output columns of the respective signal process blocks, input and output buffer names are described for each of the signal process names as an interconnection for connecting the respective signal processes. Here, since the ultrasonic scanning process (BM1) of B-mode images and the ultrasonic scanning process (DM1) of Doppler images do not need to be synchronized but may be operated simply in parallel, the process are described in the processing order using the input and output buffers as the interconnection information.

In contrast, since the TV scanning process (BM2) of B-mode images and the TV scanning process (DM2) of Doppler images involve combining the two images in the combining process, the combining process in the TV scanning process of B-mode images have two inputs (interconnection information). Moreover, since the combining process is performed on the B-mode TV scanning process side, it is necessary to wait up to the completion of the gamma correction process on the TV scanning process side of the Doppler images before performing the combining process. Therefore, a synchronization process needs to be performed before the combining process on the TV scanning process side of B-mode images and after the gamma correction process on the TV scanning process side of Doppler images.

Therefore, in the process configuration text 30, a signal process name, a synchronization process, is described before the combining process in the TV scanning process block of B-mode images, and a synchronization name sync1 is described in a sync column which is a column for describing a synchronization name corresponding to this synchronization process. Moreover, a signal process name, a synchronization process, is described after the gamma correction process in the TV scanning process block of Doppler images, and a synchronization name sync1 is described in a sync column which is a column for describing a synchronization name corresponding to this synchronization process.

Moreover, since the two processes themselves have to be processed at the same time, a synchronization process needs to be performed at the end of the TV scanning process of B-mode images and the TV scanning process of Doppler images. Therefore, in the process configuration text 30, a signal process name, a synchronization process, is described after the data transfer process in the TV scanning process block of B-mode images, and a synchronization name sync2 is described in a sync column which is a column for describing a synchronization name corresponding to this synchronization process. Moreover, a signal process name, a synchronization process, is described after the synchronization process corresponding to sync1 in the TV scanning process block of Doppler images, and a synchronization name sync2 is described in a sync column which is a column for describing a synchronization name corresponding to this synchronization process.

That is, since two synchronization processes are present in each process group of the TV scanning process block of B-mode images and the TV scanning process block of Doppler images, if they are described simply as synchronization process, the process that is to be synchronized with another process is not definite. Therefore, by adding sync attributes to the sync column of the process configuration text 30, tags are attached to the synchronization processes. In this way, in FIG. 7, synchronization processes associated with sync1 are synchronized, and synchronization processes associated with sync2 are synchronized.

According to the description of the process configuration text 30, it is possible to describe synchronization names (sync1 and sync2) of a pair of signal process names (synchronization process) which is executed in synchronization between a signal process group associated to one signal process block name (BW_Video_Process) and a signal process group associated to another signal process block name (DOP_Video_Process). Thus, signal processes associated with signal process names for which the synchronization name is described can be executed in synchronization with each other.

According to the present embodiment, by describing the signal process block name, the respective description input columns, the parenthesis { }, and the like as a format in advance, and describing only the signal process name, the interconnection information using the input and output buffer names representing the execution order of the signal processes, and the synchronization name, it is possible easily describe synchronization between parallel processes. Moreover, when it is necessary to change the signal processing configuration, it is possible to easily change the signal processing configuration without changing the source code itself of the data conversion section 20, described in a program language, for example.

In this way, for example, in a development stage of an ultrasonic diagnostic apparatus, when it is desired to verify the performance of the system while appropriately changing the signal processing configuration, it is possible to flexibly change the signal processing configuration. Moreover, since the signal processing configuration can be viewed as a text, the overall structure of the signal processing configuration can be easily grasped. As a result, the development efficiency of the ultrasonic diagnostic apparatus can be improved. Moreover, for example, in a maintenance stage of the ultrasonic diagnostic apparatus, since the signal processing configuration can be changed flexibly in accordance with a client demand or the like, the maintenance efficiency can be improved.

Particularly, when it is necessary to process different ultrasonic images in parallel and to realize synchronization between the parallel processes, it is difficult to rewrite the source codes of the program language in order to change the signal processing configuration. In this respect, as in the present embodiment, by describing a synchronization name in the process configuration text 30 with respect to signal processes executed in synchronization, it is possible to easily realize synchronization between parallel processes.

Preferred embodiments of the ultrasonic diagnostic apparatus and the like according to the invention have been described with reference to the accompanying drawings. However, the invention is not limited to the embodiments. It is clear that a person with ordinary skill in the art can easily conceive various modifications and changes within the technical idea disclosed herein, and it is contemplated that such modifications and changes naturally fall within the technical scope of the present invention.

### REFERENCE SIGNS LIST

10: ULTRASONIC DIAGNOSTIC APPARATUS

12: ULTRASONIC PROBE

14: TRANSMISSION/RECEPTION SWITCHING SECTION

16: BEAM FORMING SECTION

20: DATA CONVERSION SECTION

24: DISPLAY

20a to 20h: PROCESSOR

30: PROCESS CONFIGURATION TEXT

1. An ultrasonic diagnostic apparatus comprising:
an ultrasonic probe configured to transmit an ultrasonic beam to a subject and receive reflected echo signals from the subject;
a beam forming section configured to supply a driving signal causing the ultrasonic probe to transmit the ultrasonic beam;
a data conversion section configured to convert echo data obtained by digitalizing the reflected echo signals into ultrasonic image data; and
a display configured to display ultrasonic images based on the converted ultrasonic image data,
wherein the data conversion section executes the plurality of signal processes on the echo data based on a process configuration text of which a plurality of signal processes executed by the data conversion section and the execution order of the plurality of signal processes are rewritable in a general language, and converts the echo data into the ultrasonic image data.

2. The ultrasonic diagnostic apparatus according to claim 1,
wherein the process configuration text is configured to describe a plurality of signal process names executed by the data conversion section and an input buffer name and an output buffer name of each of the plurality of signal process names, and
wherein an output buffer name of one signal process name is made identical to an input buffer name of another signal process name to thereby describe the execution order of a signal process associated with the one signal process name and a signal process associated with the another signal process name.

3. The ultrasonic diagnostic apparatus according to claim 2,
wherein the data conversion section includes a plurality of processors,
wherein the process configuration text is configured to describe a plurality of signal process block names, a plurality of signal process names executed by the data conversion section, of each of the plurality of signal process block names, and an input buffer name and an output buffer name of each of the plurality of signal process names, and
wherein a signal process group associated with the plurality of signal process names in the respective signal process block names is allocated to the plurality of processors and executed in parallel.

4. The ultrasonic diagnostic apparatus according to claim 3,
wherein the process configuration text is configured to be capable of describing a synchronization name for a pair of signal process names which is executed in synchronization between the signal process group associated with one signal process block name and the signal process group associated with another signal process block name, and
wherein signal processes associated with a signal process name for which the synchronization name is described are executed in synchronization with each other.

5. The ultrasonic diagnostic apparatus according to claim 2,
wherein the process configuration text is configured to be capable of describing a table name for the signal process name, in which process coefficients used in signal processes are stored, and
wherein a process coefficient associated with the table name is referenced when executing a signal process associated with a signal process name for which the table name is described.

6. A signal processing method in an ultrasonic diagnostic apparatus, comprising:
a step wherein an ultrasonic probe transmits an ultrasonic beam to a subject and receives reflected echo signals from the subject;
a step wherein a beam forming section supplies a driving signal causing the ultrasonic probe to transmit the ultrasonic beam;
a step wherein a data conversion section converts echo data obtained by digitalizing the reflected echo signals into ultrasonic image data; and
a step wherein a display displays ultrasonic images based on the converted ultrasonic image data,
wherein the data conversion section executes the plurality of signal processes on the echo data based on a process configuration text of which a plurality of signal processes executed by the data conversion section and the execution order of the plurality of signal processes are rewritable in a general language, and converts the echo data into the ultrasonic image data.
